SEQUENCE LISTING (4 sequences)

SEQ ID NO: 1 - protein, 196 aa, Homo sapiens
MFCEKAMELIRELHRAPEGQLPAFNEDGLRQVLEEMKALYEQNQSDVNEAKSGGRSDLIPTIKFRHCSLLRNRRCTVAYLYDRLLRIRALRWEYGSVLPNALRFHMAAEEMEWFNNYKRSLATYMRSLGGDEGLDITQDMKPPKSLYIEVRCLKDYGEFEVDDGTSVLLKKNSQHFLPRWKCEQLIRQGVLEHILS

SEQ ID NO: 2 - protein, 185 aa, Homo sapiens
MDAAEVEFLAEKELVTIIPNFSLDKIYLIGGDLGPFNPGLPVEVPLWLAINLKQRQKCRLLPPEWMDVEKLEKMRDHERKEETFTPMPSPYYMELTKLLLNHASDNIPKADEIRTLVKDMWDTRIAKLRVSADSFVRQQEAHAKLDNLTLMEINTSGTFLTQALNHMYKLRTNLQPLESTQSQDF

SEQ ID NO: 3 - protein, 216 aa, Homo sapiens
  misc_feature (52)..(52): Xaa can be any naturally occurring amino acid
MSEAYFRVESGALGPEENFLSLDDILMSHEKLPVRTETAMPRLGAFFLERSXGAETDNAVPQGSKLELPLWLAKGLFDNKRRILSVELPKIYQEGWRTVFSADPNVVDLHKMGPHFYGFGSQLLHFDSPENADISQSLLQTFIGRFRRIMDSSQNAYNEDTSALVARLDEMERGLFQTGQKGLNDFQCWEKGQASQITASNLVQNYKKRKFTDMED

SEQ ID NO: 4 - protein, 223 aa, Homo sapiens
MTEEVDFLGQDSDGGSEEVVLTPAELIERLEQAWMNEKFAPELLESKPEIVECVMEQLEHMEENLRRAKREDLKVSIHQMEMERIRYVLSSYLRCRLMKIEKFFPHVLEKEKTRPEGEPSSLSPEELAFAREFMANTESYLKNVALKHMPPNLQKVDLFRAVPKPDLDSYVFLRVRERQENILVEPDTDEQRDYVIDLEKGSQHLIRYKTIAPLVASGAVQLI

(19) United States
(12) Patent Application Publication
(10) Pub. No.: US 2009/0053714 A1
(43) Pub. Date: Feb. 26, 2009

(54) Gins gene expression as marker for actively cycling cells and cell cycle phase

(75) Inventor: Stephen D. Bell, Cambridge (GB)

Correspondence Address: Kathleen Williams;Edwards Angell Palmer & Dodge LLP, P.O.Box 55874, Boston, MA, 02205, US

(73) Assignee: Medical Research Council, London (GB)

(21) Appl. No.: 12/075,974

(22) Filed: Mar. 14, 2008

(30) Foreign Application Priority Data
Sep. 15, 2005 (GB) PCT/GB2006/003465
Sep. 15, 2006 (GB) 0518877.6

(51) Int. Cl. C12Q 1/68

(52) U.S. Cl. 435/6

(57) ABSTRACT

The invention relates to a method of detecting an actively cycling cell in a sample, said method comprising determining the state of GINS gene expression within said cell, wherein detection of GINS gene expression in said cell indicates that said cell is actively cycling. Furthermore, the invention relates to methods for detecting an actively cycling cell in a subject, said method comprising assaying a sample from said subject for evidence of GINS gene expression, in particular when the sample is a body fluid such as urine. Preferable the GINS gene is PSFI or SLD5, in particular SLD5, in particular SLD5. The invention also relates to use of a PIKK family kinase in the in the phosphorylation of GINS, in particular when the kinase is selected from the group consisting of S. Cerevisiae Mecl and Tell, human ATR, ATM and DNA-PK.

### RELATED APPLICATIONS

This is a continuation patent application that claims priority to PCT patent application number PCT/GB2006/003465, filed on Sep. 15, 2006, which claims priority to GB patent application number 0518877.6 filed on Sep. 15, 2005, the entirety of which are herein incorporated by reference.

### FILED OF INVENTION

The invention relates to markers of ellular proliferation. In particular, the invention relates to the use of GINS proteins as markers of cancer or pre-cancerous lesions.

### BACKGROUND TO THE INVENTION

Mini chromosome maintenance porteins (MCM protins) are known as to be expressed in a cell cycle dependent maner. This property has been expoited in their use as markers of cllular proliferation. The principle is that because there porteins are known to be expressed at particular points in the cell cycle, that their detection in a population of cells is a strong indiator that those cells are actively dividing. In a diagnostic setting, visulaization of MCM porteins can be used to conveniently identfiy cells which are actively dividing against the background of quiescent of nondividing cells. In this way, cancer or pre-cancerous lesions may be identified.

WO99/21014 discloses the detection of members of the preinitiation complex of DNA replication as markers of abnormally proliferating cells or ellular growth abnormalities. WO99/21014 focusses in particular on detectino of varous MCM proteins.

However, MCM proteins are notorious for exhibiting a long lag between the recommencement of a cell cycle and their detectable expression. This can lead to difficultities in the use of MCMs in this setting. Furthermore, it may lead to “false negative” results where actively cycling cells are not detected due to this extended lag period.

Eukaryotic GINS has 4 proteins subunits, Psf1, Psf2, Psf3 and Sld5. GINS is an essential factor for DNA replication in yeast and frog systems. Xenopus GINS can be found in a large protein complex with MCM and Cdc45, but it is not known in the art whether this observation implies interaction between these proteins and GINS proteins. The mechanism of GINS action is not known in the alt.

Araki et al (Genes and Development 2003) studied MCM proteins in Xenopus. Immune precipitations led to the finding of MCM and GINS proteins as co-precipitating proteins. However, MCMs and GINS were found amongst the very large population of proteins which were precipitated in these experiments. No proof of direct interaction between MCMs and GINS can be found in the prior art. At most, Araki et al imply that GINS proteins may be associated with DNA replication.

The prior art relating to GINS arguably establishes that GINS proteins are required for the process of DNA replication to occur. Furthermore it is taught that GINS proteins are recruited to chromatin during the DNA replication initiation process. However, the prior art has numerous shortcomings in this field. For example, the human GINS proteins are not discussed in the prior art. No molecular role is established for these GINS proteins. No direct interaction partners have been identified for the GINS proteins. There is no indication whether levels of GINS proteins vary during the cell cycle. It is not known whether levels of GINS proteins are different in proliferating and non-proliferating cells. Furthermore, it is not known whether GINS proteins are involvevd in processes other than DNA replication.

Furthermore, it is established in the art that many proteins implicated in DNA replication do not vary during the cell cycle. For example, ORC complex proteins are known not to vary during the cell cycle, along with numerous other replication proteins. Therefore, there is no correlation in the art between cell cycle related regulation of expression and involvement in DNA replication.

The present invention seeks to overcome problem(s) associated with the prior art.

### SUMMARY OF THE INVENTION

The present invention is based on the surprising finding that GINS proteins are expressed at different levels within different phases of the cell cycle. Moreover, it has been found that GINS proteins are expressed at their highest levels in S-phase, that is to say they are S-phase enriched.

These findings enable the use of GINS proteins as markers of cellular proliferation. It is disclosed herein that detection of GINS protein expression correlates with participation in an active cell cycle. Thus, detection of GINS proteins may advantageously be used to indicate particular cells which are actively dividing.

Thus, in a broad aspect the present invention provides a method for detecting an actively cycling cell in a sample, said method comprising determining the state of GINS gene expression within said cell, wherein detection of GINS gene expression in said cell indicates that said cell is actively cycling.

The GINS proteins are encoded by GINS genes PSF3, PSF2, PSF1 and SLD5. Preferably the GINS gene is PSF1 or SLD5. Most preferably the GINS gene is SLD5. The technical advantage of PSF1 and SLD5, particularly SLD5, is that these two polypeptides are at the core of the GINS complex. This contributes to their greater stability. Preferably the GINS gene is not PSF2. Preferably the GINS gene is not PSF3. PSF2 and PSF3 are peripheral to the complex and therefore less suitable. These issues are discussed in more detail below.

Thus, in a preferred aspect the present invention provides a method for detecting an actively cycling cell in a sample, said method comprising determining the state of PSF1 or SLD5 gene expression within said cell, wherein detection of PSF1 or SLD5 gene expression in said cell indicates that said cell is actively cycling.

In the context of the present invention, an actively cycling cell is one which is in a state of active proliferation or division. In other words, it is a cell which is progressing through phases of the cell cycle. Quiescent cells do not progress through the cell cycle, but rather exist in a suspended ‘Gₒ’ state. Thus, quiescent cells are not actively cycling cells. Preferably an actively cycling cell is a cell which is proliferating.

In another aspect, the invention provides a method for detecting an actively cycling cell in a subject, said method comprising assaying a sample from said subject for evidence of PSF1 or SLD5 gene expression, wherein detection of PSF1 or SLD5 gene expression in said sample indicates that said subject comprises an actively cycling cell. Preferably this method is conducted in vitro. Preferably this method does not include collection of the sample from the subject.

In another aspect, the invention provides a method as described above further comprising the step of determining the state of MCM gene expression within said cell or sample, wherein detection of MCM gene expression indicates the presence of an actively cycling cell.

Presence of both GINS and MCM gene expression is advantageously a stronger indicator of the presence of an actively cycling cell than mere detection of one or the other marker alone.

In another aspect, the invention provides a method as described above wherein the sample is a body fluid and the method comprises detecting PSF1 or SLD5 protein within said body fluid.

Preferably GINS gene expression is determined by detection of GINS protein. Preferably the GINS protein, such as PSF1 or SLD5, is detected by immunochemistry.

In another aspect, the invention provides a method of identifying proliferating or non-proliferating cells in a sample said method comprising determining the state of PSF1 or SLD5 expression within said cells, wherein detection of PSF1 or SLD5 expression in a cell indicates that said cell is proliferating, and absence of PSF1 or SLD5 expression in a cell indicates that said cell is non-proliferating.

Preferably both proliferating and non-proliferating cells are detected in a single sample. Preferably the invention is used to discriminate between proliferating and non-proliferating cells in a sample, or to localise proliferating cells relative to non-proliferating cells.

The skilled reader will appreciate that although the invention is principally described in connection with positive detection of GINS, such as PSF1 or SLD5, correlating with or indicating active participation in the cell cycle, that other embodiments are also within its scope, such as the absence of GINS protein, such as PSF1 or SLD5, correlating with a non-proliferative or quiescent state. Furthermore, where embodiments are described as relating to differences in GINS expression state between cells, it should be borne in mind that a particular sample being analysed may possess only one level of GINS expression eg. absence of GINS expression in a sample of wholly non-proliferating cells. In these embodiments, it will be clear to the skilled reader that presence of a heterogeneous population of cells is not a requirement of the invention; detection of GINS expression must be considered on a cell-by-cell basis and the skilled operator is able to distinguish between GINS expression and lack of GINS expression without the need for every sample analysed to possess cells of both expression states. Preferably both GINS positive (ie. GINS expressing) and GINS negative (ie. GINS non-expressing) cells are present in each sample anaylsed, since this advantageously facilitates easy side-by-side comparison of expression states and therefore increases the reliability and readout of the assays.

In another aspect, the invention provides a method of determining the phase of the cell cycle which a cell is in, comprising determining the level of PSF1 or SLD5 protein in said cell, wherein an enhanced level of PSF1 or SLD5 protein indicates that said cell is in S-phase.

According to the present invention, in addition to being expressed in a cell cycle dependent manner, the level of GINS protein (such as PSF1 or SLD5 protein) in a cell fluctuates according to the particular phase of the cell cycle which said cell is in. In particular, GINS proteins are enriched in S-phase of the cell cycle. It will be appreciated that this enrichment is at a level above the level of GINS expression in actively cycling cells ie. above the average level of GINS expression in actively cycling cells, and therefore presence of GINS indicates cell cycle activity and furthermore, enriched presence of GINS indicates S-phase. Enriched means enhanced, elevated, augmented, boosted, increased or otherwise greater expression of GINS. Preferably this refers to presence of a greater quantity of GINS protein per cell. Naturally a reference point may be needed for accurate determination of an ‘enriched’ GINS level; a calibration reference point may be easily determined by a person skilled in the art by comparing GINS levels in actively cycling cells of interest. This may be done in a distinct population of cells of interest, and absolute values may be used to judge enrichment in a particular cell being assayed. Alternatively, the reference point may be generated by sampling and reanalysis of the population of cells being examined. In this scenario, the cells would be assayed for GINS expression. All of those cells for which expression is seen would be considered to be actively cycling. Average GINS expression can then easily be determined across that population, for example by using image analysis software on photographs of GINS immunostaining. The population of cells can then be re-examined and, using the average values for GINS expression levels amongst GINS-expressing cells, above-average expressing cells can be identified. In accordance with the present invention, these above-average GINS expressing cells are likely to be S-phase cells. Preferably the population of highest GINS expressing cells are likely to be S-phase cells. Re-examination need not involve the actual cells, for example the data or photomicrograph may simply be re-examined following the determination of average levels of GINS expression

The invention finds application in any setting in which it is desired to distinguish between dividing and non-dividing cells, and/or to determine whether a particular cell is actively cycling or not. In particular the invention finds application in diagnostic settings such as the detection of disorders of cellular proliferation. For example, the invention may be used to detect precancerous lesions and/or actual cancers. GINS expression (GINS gene expression) may be detected by any suitable means known to those skilled in the art. Expression may be detected at the nucleic acid or protein level. Detection of expression may be by mass spectrometry and assignment of the mass readouts to particular GINS protein moieties. At the nucleic acid level, detection is preferably by monitoring of mRNA levels. Preferably expression is detected at the protein level. Preferably GINS gene expression refers to GINS protein expression, preferably to PSF1 or SLD5 protein expression. Preferably GINS protein expression is determined by direct or indirect detection of GINS protein. Preferably GINS protein is detected by immunochemical means. Preferably GINS protein is detected by an antibody capable of reacting with GINS protein, and subsequent visualisation of said antibody. Preferably the antibody is a polyclonal antibody or a monoclonal antibody. Preferably when the antibody is a polyclonal antibody it is an immunopurified polyclonal antibody. Preferably the antibody is a monoclonal antibody. Use of secondary and even tertiary or further antibodies may advantageously be employed in order to amplify the signal and facilitate detection. Preferably GINS protein(s) are visualised by use of immunofluorescent means directly or indirectly bound to the GINS protein(s). Quantification of such readouts, for example in embodiments of the invention concerned with the determination of the particular phase of the cell cycle, is well within the ability of a person skilled in the art.

Preferably detection may be by ELISA or may be by Western blot.

In another aspect, the invention provides a method as described above wherein the detection is performed on a liquid sample.

In another aspect, the invention provides a method as described above wherein the Psf1 or Sld5 is extracellular.

Preferred reagents for GINS detection include the commercially available hPSF2 antibody from Genway (catalogue number 15-288-22115F); published anti-Xenopus Psf3 antisera (Kubota et al., 2003 Genes Dev. vol 17 pages 1141-1152 (e.g. by cross reaction with other species such as human)); or any other reagent capable of binding or reading out presence of GINS proteins, such as antibodies against the GINS proteins produced as described herein, preferably Sld5 and/or Psf1.

In another aspect, the invention provides the use of a PIKK family kinase in the phosphorylation of GINS. Preferably said kinase is selected from the group consisting of S. cerevisiae Mec1 and Tel1, human ATR, ATM and DNA-PK More preferably said kinase is selected from the group consisting of human ATR, ATM and DNA-PK.

### DETAILED DESCRIPTION OF THE INVENTION

In order to accurately diagnose diseases of cellular proliferation, such as cancer, it is advantageous to accurately determine the status or level of cell division in comparison with normal tissue. We have established that the DNA replication associated proteins of the GINS complex are present at high levels in tissue that is actively dividing. By assaying GINS expression, particularly using specific antibodies generated against GINS components in immunochemical, such as immunohistochemical, methodologies, it is possible to directly visualise potentially cancerous tissue. This allows a rapid and qualitative discrimination between potentially cancerous and normal tissue. Thus the use of antibodies against GINS can serve as an early detection system for cancer and pre-cancerous conditions, or any condition for which proliferation correlates with a disease state. In other words, the invention finds application in any setting in which a diagnosis or prognosis can be aided by in indication of the proliferative state of cells in a subject being examined.

The GINS proteins are smaller than the MCM proteins. The GINS proteins are preferably more stable than the MCM proteins. Therefore GINS proteins offer advantages in terms of provision of a more robust marker. GINS proteins may be more readily detectable in body fluids than the MCM proteins.

Moreover, the spectrum of tissue and tumour types that the GINS antisera are effective against appears to offer a different profile to MCM, affording a greater useful flexibility than the MCM proteins, and lending further advantage to the combinatorial aspects of the present invention, such as dual or parallel typing with GINS and MCM markers. In particular the complementarity between GINS and other markers may be advantageous in covering a broad spectrum of conditions with a two-fold marker analysis, which coverage cannot be achieved by use of two prior art markers such as two MCM markers together.

Thus the diagnostic and prognostic benefits of the GINS antisera apply to a broad range of tissue and tumour types.

It is disclosed herein that GINS is as effective a marker as MCM. This alone establishes the industrial application of the invention as an extremely attractive marker for commercial exploitation. This may be applied as an alternative to MCMs or other markers. This may also be applied in combination with MCMs or other markers, in particular to provide complementary coverage between different spectra of marker.

As noted above, GINS may be more stable and/or have a different diagnostic or prognostic potential from MCMs. For at least these reasons, GINS may be an advantageous marker compared to MCM.

GINS Proteins

GINS proteins are not members of the pre-replicative complex. However, it is surprisingly disclosed herein that GINS proteins can be physically associated with members of the pre-replicative complex of DNA replication in vivo.

This is particularly surprising given that GINS is unrelated in sequence and structure to all other replication associated proteins, including MCM proteins. Furthermore, replication associated proteins are not necessarily up-regulated in proliferating cells, there are numerous such proteins which show no cell-cycle related shifts in expression pattern. Thus, in the absence of the disclosures of the present invention, there is no reason to consider that expression of GINS proteins would be cell-cycle regulated.

It is surprisingly disclosed herein that GINS has a central role at the replication fork, and that levels of GINS components are regulated and different in cycling cells compared to non-cycling cells.

Moreover, the inventors show for the first time that archaeal GINS interacts directly with MCM, and more importantly that human GINS components interact directly with human MCM components.

It is disclosed herein that GINS protein levels, such as Psf1 or Sld5 protein levels, are elevated in proliferating cells. It is further shown that levels are particularly enriched in S-phase of proliferating cells.

It is also shown that GINS is a central nexus in the replication fork, coordinating leading and lagging strand synthesis, and data from archaea are presented.

These aspects of the invention are discussed in more detail in the examples section, together with demonstrations of GINS proteins being used as markers for proliferation.

The GINS complex proteins are Psf3, Psf2, Psf1 and Sld5. The sequences of the human GINS genes and their polypeptides are known in the art. Preferred GINS proteins are Sld5 and Psf1. Most preferred is Sld5.

Psf1 and Sld5 are preferred, since they provide numerous technical benefits as disclosed herein. These preferred proteins are part of the core GINS complex, whereas Psf2 and Psf3 are peripheral components and may not be as tightly regulated. Furthermore, Sld5 and Psf1 being at the heart of the complex they are more likely to be regulatory targets and thus may provide further information as well as being more biologically relevant. In addition, we have shown that Sld5 and Psf1 form a more stable subcomplex within the overall GINS complex. Thus, the fact that they are intimately associated in vivo also makes them a more attractive target for detection according to the present invention, and makes them easier to work with, thereby saving labour and costs.

Sld5 and Psf1 produce the two best immune responses in antibody generation against the four individual GINS proteins by established procedures. Thus, these two proteins are preferred according to the present invention for this advantageous feature.

Sld5 is most preferred, the technical benefit is that Sld5 expression is restricted to proliferating cells. Furthermore, Sld5 shows the best immune response in antibody generation as described herein (by standard techniques known in the art). Moreover, Sld5 is the largest of the GINS proteins and so offers more material or a larger target for detection.

Preferably the GINS protein is not Psf3 since Psf3 may persist in some differentiated tissue after division has stopped due to a slower decay rate, which may cause results to be more difficult to interpret. Psf3 may find application as an extracellular marker. Thus, when the GINS protein is Psf3, preferably detection is of extracellular Psf3 protein.

Preferably the GINS protein is not Psf2 or Psf3 since these two GINS proteins contain SQ/TQ/SQE motifs. These are known targets for phosphorylation in response to DNA damage. This event will not only complicate matters in terms of interpretation of results (e.g. differential detection of phosphorylated and unphosphorylated species), but more significantly will alter epitopes in the phosphorylated and unphosphorylated states, complicating antibody generation and perhaps masking other epitopes due to conformational change. Furthermore, we have shown interaction of Psf2/3 with MCM proteins in archeal systems. This may lead to masking of epitopes and therefore make Psf2/3 less useful targets for detection Thus, preferably the GINS protein is not Psf2; preferably the GINS protein is not Psf3.

Use of or detection of GINS proteins according to the present invention has the advantage that fluid detection such as liquid detection is possible. Such detection is not possible with prior art markers such as MCM. Thus, preferably the sample comprises fluid, preferably liquid. Preferably the liquid is or is derived from lysed cells, or a body fluid such as serum or urine. Preferably the liquid is or is derived from serum or urine.

In another embodiment, preferably the sample analysed is a solid phase sample such as a blot or other immobilised material. This has the advantage that washing or manipulation of the sample can be facilitated when it is in the solid phase. Of course, a solid phase sample for analysis may be created from a liquid phase starting sample, e.g. by size separating the liquid sample and immobilising it such as by Western blotting. Preferably in liquid detection embodiments, the detection is directly carried out on a liquid sample, for example by placing the liquid sample in an ELISA well precoated with an anti-GINS antibody (followed by appropriate washing/handling/detection).

It is a drawback of MCM that extracellular detection does not work, as has been documented in the art. By contrast, GINS proteins find application as extracellular markers for proliferation. Thus, detection in material from lysed cells or other liquid modes of detection may advantageously be employed in accordance with the present invention. Particularly preferred liquid detection is detection from serum and/or detection from urine.

It is demonstrated herein that GINS proteins are advantageously stable. Indeed, GINS proteins show resistance to degradation in comparison with MCM. This is particularly advantageous for Sld5 and Psf1.

GINS proteins such as Psf1 and Sld5 perform better than MCM in assays, and are smaller and more stable. Without wishing to be bound by theory, it is possible that these advantages may be attached to their small and globular nature.

Preferred GINS sequences are given below:

Psf1 Accession No. Q14691
MFCEKAMELIRELHRAPEGQLPAFNEDGLRQVLEEMKALYEQNQSDVNEAKSGGRSDLIPTIKFRHCSLLRNRRCTV
AYLYDRLLRIRALRWEYGSVLPNALRFHMAAEEMEWFNNYKRSLATYMRSLGGDEGLDITQDMKPPKSLYIEVRCLK
DYGEFEVDDGTSVLLKKNSQHFLPRWKCEQLIRQGVLEHILS*
Psf2 Accession No. Q9Y248
MDAAEVEFLAEKELVTIIPNFSLDKIYLIGGDLGPFNPGLPVEVPLWLAINLKQRQKCRLLPPEWMDVEKLEKMRDH
ERKEETFTPMPSPYYMELTKLLLNHASDNIPKADEIRTLVKDMWDTRIAKLRVSADSFVRQQEAHAKLDNLTLMEIN
TSGTFLTQALNHMYKLRTNLQPLESTQSQDF*
Psf3 Accession No. AAH14437
MSEAYFRVESGALGPEENFLSLDDILMSHEKLPVRTETAMPRLGAFFLERSXGAETDNAVPQGSKLELPLWLAKGLF
DNKRRILSVELPKIYQEGWRTVFSADPNVVDLHKMGPHFYGFGSQLLHFDSPENADISQSLLQTFIGRFRRIMDSSQ
NAYNEDTSALVARLDEMERGLFQTGQKGLNDFQCWEKGQASQITASNLVQNYKKRKFTDMED*
Sld5 Accession No. AAH05995
MTEEVDFLGQDSDGGSEEVVLTPAELIERLEQAWMNEKFAPELLESKPEIVECVMEQLEHMEENLRRAKREDLKVSI
HQMEMERIRYVLSSYLRCRLMKIEKFFPHVLEKEKTRPEGEPSSLSPEELAFAREFMANTESYLKNVALKHMPPNLQ
KVDLFRAVPKPDLDSYVFLRVRERQENILVEPDTDEQRDYVIDLEKGSQHLIRYKTIAPLVASGAVQLI*

Sample

The sample may be individual cell(s), may be a tissue biopsy or may be any other suitable material in which GINS expression eg. GINS proteins can be detected such as faeces, urine, or protein recovered from urine or other suitable material. Preferably the sample is a biopsy, which has the advantage that histological information can be added to the GINS readout, thereby bolstering the results of a test according to the present invention. In another embodiment, body fluid such as urine is a preferred sample, which offers the advantage that it is easily collected from a subject in a non-invasive manner. When detection of GINS is by detection of GINS protein, it may be advantageous to pre-extract the proteins from the sample, for example by protein recovery when the sample is urine, in order to facilitate handling and/or detection when protein levels in the sample can be low. Preferably the sample is protein recovered from urine. In a preferred embodiment, such a sample is analysed using a matrix-antibody capture system to extract protein from the sample by trapping of the antigen, followed by mass spectrometry to identify the GINS protein (if any is present). Practising the invention on samples comprising body fluids is an advantage of the present invention made possible by the greater stability of GINS in such body fluids as compared to prior art markers such as MCM proteins.

The sample may be cervix (either smear sample or biopsy), breast, colon, lung, bladder, skin, oesophagus, larynx, bronchus, lymph node, urinary tract (either biopsy or cytology smear), brushings such as brushings from the alimentary canal or oesophagus, or cells collected from urine, blood, serum or other body fluid, or may be a body fluid per se such as urine, or material extracted therefrom such as proteins from lysed cells or proteins from urine or any other suitable sample which can be tested for GINS expression.

Combinations

GINS protein detection may be advantageously combined with detection of other markers of cellular proliferation such as MCM (minichromosome maintenance) proteins, and/or geminin. Preferably GINS protein detection is combined with detection of MCM proteins. Preferably GINS protein detection is combined with one or more of Cdc6, MCM2, MCM3, MCM4, MCM5, MCM6, MCM7 or MCM8.

Advantageously, GINS may behave as a complementary marker rather than identically to MCM and/or geminin, thereby offering distinct prognostic predictive power from that of MCM or geminin.

GINS protein detection may be advantageously combined with cell type markers, for example to distinguish proliferating and non-proliferating cells of a particular tissue type. For example, it may be advantageous to combine GINS detection with detection of markers for squamous or columnar epithelium when analysing a sample from a patient suspected of having Barrett's oesophagus, advantageously allowing a measure of proliferation to be combined with an indication of the actual cell type which is proliferating, which can aid diagnosis and/or prognosis. In any case, whenever the invention is applied to distinguish proliferating from non-proliferating cells in a sample from a subject, or simply to detect the presence of proliferating cells in a subject, then the detection of proliferating cells itself aids diagnosis (and may advantageously aid prognosis) by providing a positive indication of the presence or absence of proliferating cells to the operator.

The invention will now be described by way of example which are intended to be illustrative and not limiting in nature.

### BRIEF DESCRIPTION OF THE DRAWINGS

FIG. 1 shows a western blot indicating relative levels of a GINS component, Psf3, in cycling and quiescent cells.

FIG. 2 shows photomicrographs showing staining (dark brown) of anti-Psf3 antisera in proliferating cells in cervical and colon cancer and a high grade lesion of the cervix. Normal tissue shows much fainter background staining.

FIG. 3 shows a western blot indicating relative levels of GINS in different phases of the cell cycle, and its absence from quiescent cells, using anti-Psf2 antisera.

FIG. 4 shows a western blot indicating relative levels of preferred GINS proteins Psf1 and Sld5 in replicating and non-replicating cells.

FIG. 5 shows detection of preferred GINS protein Sld5 in proliferating cells (dark or brown staining). Normal (quiescent or non-dividing) tissue shows only faint background staining.

FIG. 6 shows a western blot demonstrating protease stability of GINS such as Sld5 compared with protease sensitivity of MCM such as Mcm2. ‘−’ indicates no protease. The upper and lower panels are exactly the same blot, cleaned and reprobed with the appropriate antibody as marked.

FIG. 7 shows a skin stain with squamous cell carcinoma compared to normal tissue.

### EXAMPLE 1

### Identification of GINS Proteins as MCM Interactors

Overview

In the course of studies to understand the interplay of the DNA replication proteins we utilised yeast 2-hybrid analysis to map interactions between target genes and to identify novel interactors in screens of a genomic DNA library. We have thus identified an archaeal homologue of the eukaryotic GINS complex. The GINS complex is an essential DNA replication factor that is required for the establishment and maintenance of replication forks in budding yeast. Cells deficient in GINS are compromised in their ability to recruit DNA polymerase in replication initiation. The GINS complex also appears to be required for recruitment of the replicative DNA polymerase in Xenopus. However, the molecular mechanisms of action of GINS have not been well understood in the prior art. Immunoprecipitation data indicate that GINS may be in some way associated with the MCM complex in Xenopus. Whether this interaction is direct or indirect is not known in the art. We disclose information on the function of this poorly understood yet essential eukaryotic replication factor, and aspects of the present invention are based on these findings.

Interaction Screen

In a 2-hybrid screen using the S. solfataricus MCM as bait we identified a novel interactor that upon PsiBlast analysis was revealed to be homologous to the Psf2 component of human GINS, hereafter this will referred to as ‘ssGINS’. All four components of eukaryotic GINS are derived from a common ancestor and the archaeal GINS homologue we have identified is closely related to that ancestor.

We have performed deletion analysis in the 2-hybrid assay and found that the N-terminal domain of MCM interacts specifically with GINS. In addition, we have expressed GINS as recombinant protein and demonstrated a direct interaction between MCM and ssGINS in GST-pull down experiments.

Mapping of the MCM-GINS interaction reveals that GINS interacts with the N-terminal half of MCM.

Analytical ultracentrifugation and gel filtration analyses have revealed that S. solfataricus MCM is predominantly a hexamer in solution. Eukaryotic GINS is most likely a heterotetramer. Analytical ultracentrifugation and gel filtration on purified recombinant ssGINS is used to establish its multimeric status.

### EXAMPLE 2

### Examination of Human GINS-MCM Interaction

Overview

In Xenopus, MCM co-immunoprecipitates with GINS. It is unknown in the art whether this is a direct or indirect interaction. In light of our data on the archaeal GINS-MCM interaction, we disclose that the interaction is direct. We use the yeast two-hybrid approach to show this by looking for direct interactions between individual MCM subunits and GINS subunits.

Interaction Analysis

DNA binding domain and activation domain fusion constructs for all six human MCM subunits are as described in Yu et al, (2004) J. Mol. Biol, 340, 1197-1206 (supplied by from Dr. C. Liang (PR China)). The human GINS subunits are cloned into the appropriate vectors.

Interactions are detected and the information gleaned from our analysis of the archaeal GINS-MCM interaction is used to map the precise interaction sites. In this way, the interaction interface can be narrowed down to a sufficiently small region, and peptides corresponding to this region of MCM and GINS subunits are synthesised.

The ability of these peptides to interfere with DNA replication in the human cell free in vitro DNA replication system is tested. Thus, the invention relates to the use of peptides involved in the MCM-GINS interaction in the modulation of DNA replication. Preferably said peptides are GINS peptides. Preferably said peptides inhibit DNA replication and are thus useful as cell proliferation inhibitors for the control of disorders of cellular proliferation.

### EXAMPLE 3

### GINS Proteins and DNA Repair

The sequences of higher eukaryotic GINS components contain conserved multiple SQ and TQ dipeptides, corresponding to the phosphorylation site preference of the phosphatidylinositol-3 kinase-like kinases (PIKK) family of kinases (including ATR, ATM and DNA-PK; mutations in these Idnases have cancer predisposition phenotypes in vertebrates). We disclose that the GINS motifs may represent target sites for these damage-sensing kinases. We further disclose that, given the established role for eukaryotic GINS in replication progression, the PIKK kinases may phosphorylate GINS in response to stalling of replication forks. Thus the invention relates to modulation of DNA replication progression by phosphorylation of GINS protein. Preferably said phosphorylation is by a kinase selected from the group consisting of S. cerevisiae Mec1 and Tel1, human ATR, ATM and DNA-PK, preferably human ATR, ATM and DNA-PK. Furthermore, the invention relates to inhibition of a PIKK family kinase by a GINS peptide. Preferably said peptide comprises a TQ and/or SQ dipeptide. Preferably said peptide comprises GINS sequence surrounding the naturally occurring TQ/SQ dipeptides. Preferably said peptide is at least 8 amino acids long, preferably at least 10, preferably at least 15, preferably at least 20, preferably at least 40 amino acids long. Preferably the TQ/SQ dipeptides are located in the middle of the peptide. Combinatorial peptides may advantageously be used, for example by concatenating peptides according to the present invention. Clearly in this embodiment, the total size of the peptide will be correspondingly larger and the TQ/SQ sites may be dispersed in the peptide, for example at one quarter and three quarter positions for a two-peptide concatenated combination.

Genetic Analysis of GINS in S. cerevisiae

Yeast GINS subunits, Psf1 and Sld5 also contain SQ and TQ residues. Site-directed mutagenesis is performed to change the serine and threonine residues to alanine, introduce these mutated GINS into yeast cells in which the chromosomal copy of PSF1 and SLD5 have been deleted and viability supported by episomal copies of wild-type PSFI and SLD5 on a URA3 containing plasmid. Plasmid shuffling is then performed to introduce the SQ/TQ mutated alleles and the growth of the new strains monitored in the presence of a number of DNA damaging agents for example hydroxyurea, which is known to result in stalled replication forks. The results implicate the SQ and TQ in DNA damage responses, and so epitope tagged Psf1 and Sld5 are expressed in yeast cells and mobility of the proteins before and after the appropriate stimuli is assayed by SDS-PAGE to test for covalent modification. Covalent modification in this setting demonstrates a role of the SQ and TQ in damage response. The kinase(s) responsible are identified using genetic assays, with the focus on the yeast PIKKs Mec1 and Tel1.

Thus the invention relates to the use of a PIKK family kinase in the phosphorylation of GINS, preferably said kinase is selected from the group consisting of S. cerevisiae Mec1 and Tel1, human ATR, ATM and DNA-PK, preferably said kinase is selected from the group consisting of human ATR, ATM and DNA-PK.

### EXAMPLE 4

### Human GINS

Antisera are raised against purified human GINS subunits.

GINS protein purification and antiserum production

The open reading frames (ORFs) for the GINS subunits are cloned into the pET series of bacterial expression vectors. The ORFs lack stop codons and so are translationally fused to a hexa-histidine encoding 3′ extension. Thus, the proteins produced by these vectors have a 6-His tag at the C-terminus.

BL21 Rosetta cells containing the appropriate expression construct are grown in a 50 ml overnight culture of L-Broth, supplemented with 40 μg/mL kanamycin and 34 μg/ml chloramphenicol. Growth is at 37° C. at 200 rpm. Next morning 20 ml of the overnight culture is diluted into 1 litre of fresh, pre-heated L-Broth and supplemented with kanamycin and chloramphenicol to 40 μg/ml 34 μg/ml respectively. Cells are grown at 37° C. at 200 rpm to OD600 nm=0.5 and IPTG is then added to 1 mM to induce expression. Continue induction for 4 hours. Harvest cells by centrifugation, and resuspend the cell pellet in 25 ml of Buffer A (10 mM Tris, pH8.0, 150 mM NaCl). Lyse the cells by passage through a Emulsiflex cell disruptor. Centrifuge lysate at 40,000 g. The GINS protein(s) of interest (Psf1, Psf2, Psf3, Sld5) will be pelleted. Discard the supernatant and resuspend the pellet in Buffer A containing 8M urea. Centrifuge this material at 40,000 g and recover the supernatant. The GINS proteins are now soluble and present in the supernatant.

Prepare a 1 ml bed volume Ni-NTA agarose (Qiagen) column in a 20 cm×1 cm diameter Sigma column. Equilibrate the matrix with 20 ml of Buffer A+8M Urea using gravity flow. Apply the GINS protein-containing supernatant to the column by gravity flow, retain 10 μl for subsequent analysis (designated IN). The GINS protein of interest is retained on the column. Collect the flow through material-designated FT. Wash the column with 20 ml of Buffer A +8M urea. Collect the material flowing through the column- designated W1. Wash the column by gravity flow with 10 ml of Buffer A+8M urea+20 mM imidazole. Collect the material flowing through the column-designated W2. Elute the bound protein by applying 10 ml of Buffer A+8M urea+500 mM imidazole. Collect 1 ml fractions, designated EL 1-10. Analyse 10 μl samples of the IN, FT, W1, W2, ELI-10 by SDS-PAGE and stain gel with Coomassie brilliant blue. Fractions containing the required protein are pooled.

Immunisation for production of antiserum is carried out according to standard techniques. For generation of rabbit polyclonal antiserum, 3 volumes of Buffer A are added to the eluted GINS protein prior to immunisation of rabbits.

For affinity purification of the polyclonal antibodies, purified GINS proteins are first passed over PD10 desalting column (Amersham Biosciences), to exchange urea for SDS, before immobilising them on a SulfoLink column (Pierce) as per the manufacturer's instructions. Antisera are affinity purified following the procedure described in Harlow and Lane, Antibodies, CSH Press.

### EXAMPLE 5

### Characterisation of GINS Function

The electrophoretic mobility of GINS components is tested before and after genotoxic insult in order to evidence the modification of GINS in response to said insult.

Evidence for damage or fork stalling dependent modification of GINS leads to determination of the association of GINS with replication foci following damage using immunofluorescence.

The ability of human GINS to co-immunopreciptitate with components of the replication machinery is tested before and after treatment with genotoxic agents.

Immunoprecipitation/kinase assays are performed to identify the kinase(s) responsible for phosphorylation of GINS components, and to distinguish their relative individual involvement focussing on ATR, ATM and DNA-PK.

### EXAMPLE 6

### Use of Human GINS as Marker for Proliferation

Overview

Our analysis of archaeal GINS provides insight into the function of this poorly characterised, yet essential, DNA replication protein and serves to inform with regard to human GINS. In this example we demonstrate use of GINS as a marker for human proliferative disease, eg. as a marker for cellular proliferation and pre-cancerous conditions. The conservation of the GINS-MCM interaction over the 2 billion year evolutionary gulf between Xenopus and archaea suggests a fundamentally important functional relevance to this interaction. Thus, GINS levels serve as a discriminatory marker between proliferative and non-proliferative cells.

GINS Determination

Recombinant human GINS subunits are produced and purified for use as antigens to raise antisera as described above. It is determined whether the presence of GINS subunits correlates with the proliferative status of cells.

This example shows a method for detecting an actively cycling cell in a sample. In this case the samples of cells are lysed and their proteins extracted in order to determine the state of GINS gene expression within the cells.

Proteins are extracted from cycling and quiescent cells. The proteins are size separated by SDS-PAGE. The size separated proteins are then Western blotted onto a carrier membrane and probed using anti-GINS antibody. Actin protein is also visualised as a control to establish equivalent amounts of total protein in the cycling and non-cycling cell treatments.

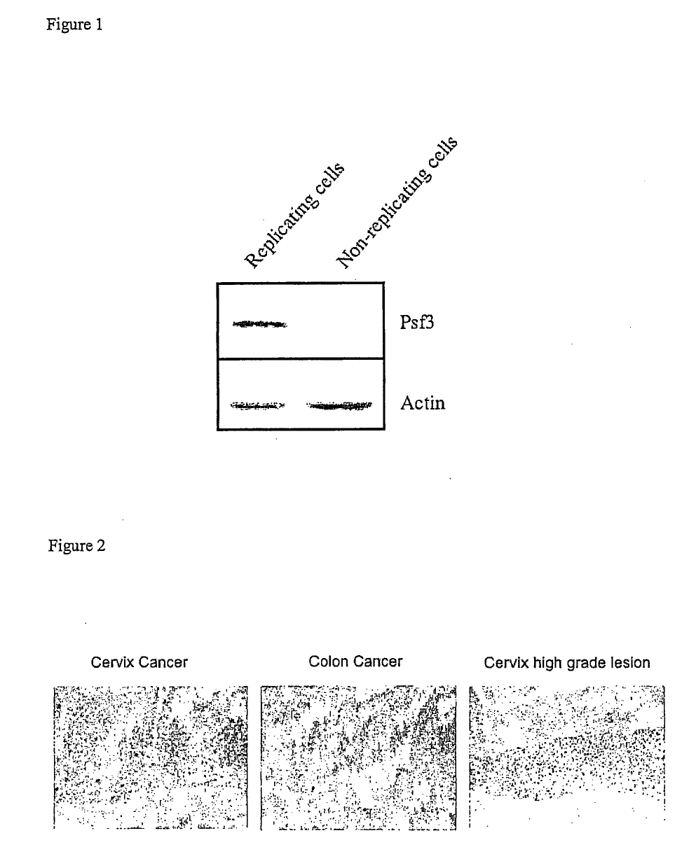

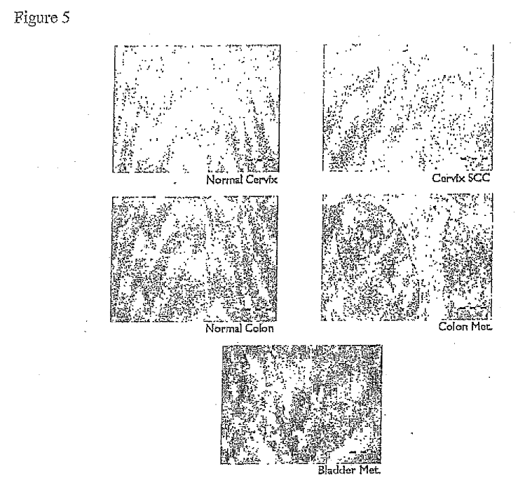

FIG. 1 shows a western blot indicating relative levels of a GINS component, Psf3, in cycling and quiescent cells. Actin serves as a loading control. GINS (Psf3) is clearly more abundant in replicating cells. FIG. 5 shows this for the preferred GINS proteins Sld5 and Psf1.

Therefore detection of GINS gene expression in the cells indicates that those cells are actively cycling.

Thus it is demonstrated that GINS is an effective marker correlating with cell proliferation.

### EXAMPLE 7

### Use of GINS in Diagnosis of Cancer and Precancerous Lesion

This example relates to a method of identifying proliferating or non-proliferating cells in a sample. This identification is performed by determining the state of GINS expression within said cells.

In this example, the state of GINS expression within said cells is determined by immunohistochemistry using the antibodies generated as described above.

Tissue biopsies are taken from a subject to be investigated. Preferably taking of the samples is not a part of the present invention; in this example the samples are provided as the in vitro start point for the methods of the invention.

The biopsies are sectioned and fixed by conventional methods to allow immunological visualisation of GINS using anti-GINS antibody as described above. In this example the anti-GINS antibody is anti-Psf3 antibody. The anti-GINS antibody is applied to the samples, allowed to bind, excess is washed away, secondary antibody is applied to visually stain the bound primary anti-GINS antibody and photomicrograhs of the samples are produced as shown in FIG. 2. FIG. 2 shows staining (dark areas; dark brown in colour reproductions) of anti-Psf3 antisera in proliferating cells in cervical and colon cancer and a high grade lesion of the cervix. Normal tissue shows extremely faint background staining, indicative of the absence of GINS expression. FIG. 5 shows this for preferred GINS protein Sld5.

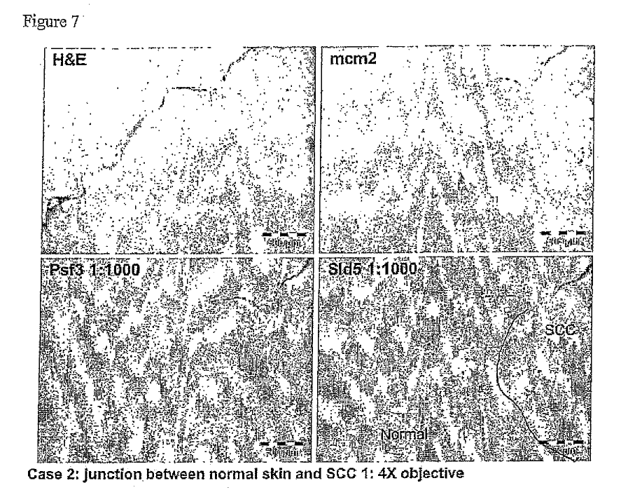

FIG. 7 shows a skin stain with squamous cell carcinoma compared to normal tissue. It can be clearly seen that the proliferating cells of the squamous cell carcinoma stain heavily with reagent for detection of GINS protein according to the present invention. In particular, preferred GINS protein Sld5 staining is shown in the bottom right panel.

Thus, detection of GINS expression in a cell indicates that said cell is proliferating, and absence of GINS expression in a cell indicates that said cell is non-proliferating.

In this example, GINS markers have been used to distinguish abnormal cellular proliferation in cancer and pre-cancer lesions from the surrounding non-proliferative, quiescent (ie. healthy or normal) tissue.

In this way, the diagnosis of a cell proliferation disorder is aided.

### EXAMPLE 8

### Enriched GINS Indicates S-Phase

As is explained above in detail, Go vs cycling cells can be distinguished reliably and sensitively by presence or absence of GINS protein. In this example, we show a further benefit of the invention in that by comparing GINS protein levels, S-phase enrichment of GINS can be observed. Thus, the invention relates to the identification of S-phase cells by detection of enriched GINS protein levels, ie. detection of enriched GINS protein indicates the cell(s) are in S-phase.

Embryonic fibroblast cells enriched in G1 and S phase were prepared by standard methods. The extracts were prepared as detailed in Krude et al. 1997 (Krude, T., Jackman M., Pines, J. and Laskey R. A. Cyclin/Cdk-Dependent Initiation of DNA Replication in a Human Cell-Free System 1997 88: 109-119). Briefly, the preparation described is as follows:

To prepare S phase nuclei or extracts, cells were synchronized in S phase by a single block in culture medium containing 2.5 mM thymidine (Sigma) for 25 hr, followed by a release into culture medium for 2 hr.

Cells in G1 phase were obtained by releasing cells blocked in very early S phase into culture medium for 3 hr, followed by adding 40 ng/ml nocodazole (Sigma) for an additional 12 hr to arrest them in mitosis. These mitotic cells were then released into fresh culture medium for 6 hr unless otherwise indicated.

Whole cell protein extract was then prepared, size fractionated by gel electrophoresis and western blotted onto a suitable support.

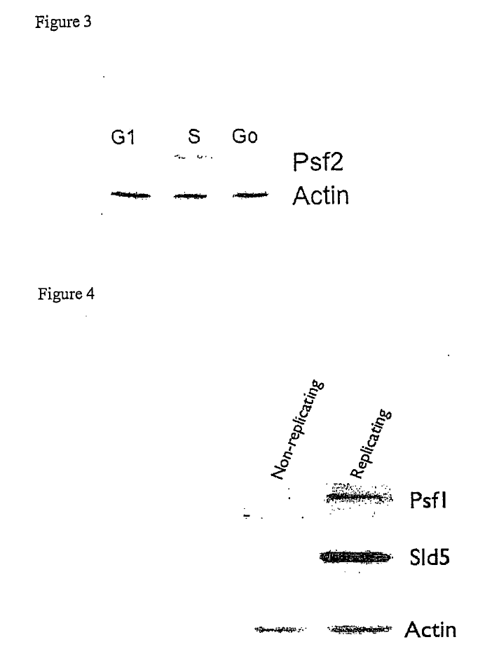

FIG. 3 shows detection of Psf2 protein using anti-Psf2 antibody produced as described above (see example 4). The S-phase enrichment of GINS protein is clearly demonstrated.

At normal length exposures, GINS protein is shown to be present in G1 cells. FIG. 3 shows an especially short exposure in order to illustrate the strength of the S-phase enrichment. It should be noted that GINS protein is always absent from quiescent (Go) cells so that the main focus of the invention of distinguishing between Go and cycling cells is not affected by the extra benefits of assaying for S-phase enrichment. In other words, presence of GINS protein correlates with cycling cells. The apparent absence of GINS protein in the G1 treatment of FIG. 3 is created by the unusually short exposure used in this example to demonstrate the S-phase enrichment. Under ordinary exposures, GINS is present in S-phase and G1 cells, but not Go cells, and thus functions to distinguish cycling from non-cycling cells as discussed herein.

### EXAMPLE 9

### Detection by Immunoblotting

The detection method of this example may be applied to any suitable sample. Samples, resolved on 12% acrylamide gel by SDS-PAGE, were blotted onto a nitrocellulose membrane and blocked in 5% (w/v) marvel, TBST (10 mM Tris-HCl pH 8.0, 150 mM NaCal, 0.1% (v/v) Tween-20) overnight at 4° C. Primary antisera (diluted to 0.1% (v/v) in TBST) were detected using horseradish peroxidase-coupled anti-rabbit antibodies (Pierce) diluted to 0.01% (v/v) in TBST. The blots were developed using ECL Western blotting detection system (Amersham Biosciences).

### EXAMPLE 10

### Detection by ELISA

The detection method of this example may be applied to any suitable sample, particularly liquid sample(s).

ELISA plate (96-well, Nunc) was coated with 100 μl 20 μg/ml purified Sld5 antisera in PBS (137 mM NaCl, 2.7 mM KCl, 8 mM Na2HPO4, 2 mM KH2PO4, pH 7.4) at 4° C. overnight. After two washes with PBS, wells were blocked with the blocking buffer (PBS, 3% (w/v) bovine serum albumin, 0.05% thimerosal) at 4° C. overnight. After two washes with PBS-T (PBS, 0.1% (v/v) Tween-20), wells were incubated with 100 μl samples of appropriately processed human fluid samples or Sld5 protein standards for at least 2 h at room temperature. After the PBS-T wash (four washes), wells were incubated with 100 μl of the purified Sld5 antisera diluted in PBS-T for 2 h at room temperature and the washed again, incubated with 200 μl secondary antibody solution (ImmunoPure Goat Anti-Rabbit IgG, Peroxidase Conjugated, Pierce diluted in PBS-T according to manufacturer's recommendations) for two hours at room temperature and washed again. Freshly-prepared substrate solution (100 μl; 0.1% (w/v) 3′,3′,5′,5′-Tetramethylbenzidine, 0.1 M sodium acetate, 0.01% hydrogen peroxide) was added to the wells and the reaction was allowed to proceed in the dark at room temperature until blue product was visible (between 10 and 60 min). The reaction was stopped by the addition of 50 μl 1 M H2SO4. The absorbance was analysed using the Fusion microplate reader Fusion set at 450 nm.

### EXAMPLE 11

### Handling and Preparation of Serum Samples

Serum samples are preferably prepared as detailed in this example. Serum samples may then be assayed directly (liquid detection) or by converting into a solid phase sample (e.g. by blotting as in example 9).

Protocol for Handling and Centrifugation of Blood Samples for the Collection of Serum

Equipment: Centrifuge-Sorvall Legend T with containment screw caps for centrifuge carriages

P1000 and P200 pipettes and tips

Trigene™ 5%—Antibacterial, antifungal and virucidal spray

Tissue paper

Personal protective equipment (PPE)—Laboratory coat and gloves

Tube rack suitable for 15 ml tubes

Green top (Coagulation 9 NC 10 ml) blood tubes

Codes of Practice for a Category 2 laboratory should be followed.

Protocol:

Paired blood samples arrive (e.g. by post) in plastic blood bottles within purpose designed blood transport tubes

1 sample in red top (EDTA KE 9 ml) tube—Frozen on arrival at −80oC

1 sample in green top (Coagulation 9 NC 10 ml) tube—Stored at room temperature prior to centrifugation

Tubes stored unopened in sealed polystyrene storage container

Blood samples moved to Category 2 containment room

Pre-centrifugation checks undertaken e.g. ensure centrifuge is switched on, each internal carriage has screw top lid present and no samples or other material has been left by previous users

Label appropriate number of green top (Coagulation 9 NC 10 ml) blood collection tubes with unique identifier matching that recorded on samples to be centrifuged.

Start Class 2 hood, store hood night door safely and ensure air velocity on indicator panel reaches 0.45-0.55 ms-1

Spray the interior of the hood using Trigene 5%, wipe using tissue paper and dispose of paper in yellow incineration bin.

Open centrifuge cover using unlocking button

Raise and support cover ensuring that it does not fall downwards and risk injury

Remove screw tops from centrifuge carriages to be used

Place tubes into the centrifuge carriage, ensuring that they are arranged to balance each other, if necessary use additional spare tubes filled with water to balance centrifuge.

Replace screw top carriage covers, ensuring that they are firmly closed

Carefully lower centrifuge cover until closed. NB Final 1 cm of closure assisted automatically by locking mechanism, talce care to avoid trapping fingers or equipment during this procedure

Set centrifuge to spin at 3000 rpm for 10 minutes

When complete, unlock centrifuge cover, carefully raise cover

Unscrew carriage covers and carefully examine blood bottles to assess whether separation of serum phase has occurred. Avoid agitation at this point to prevent mixing of serum layer with cellular layer

If separation has occurred move sample into the hood with a similarly labeled tube in a tube rack

If separation has not occurred it is likely that red cell haemolysis has occurred, store this sample in its unseparated state at −80 recording in the database that separation failed to occur

Ensuring that appropriate PPE is worn, within the hood remove the screw top from the recently spun blood bottle.

Take the matched labelled bottle for serum collection, ensure that the plunger has been fully withdrawn and locked, snap off the plunger

Unscrew the top from the collection bottle

Take P1000 or P200 pipette and filter sealed pipette tips and carefully draw up serum (Upper yellow layer of fluid), place collected serum in new matched tube.

Ensure that none of the lower cellular layer is drawn up during this process

When each sample is separated replace the screw caps and place in a clip sealed bag for freezing

After each pipette tip is used dispose in the incineration bin within the extractor hood

When complete spray down interior of hood and all hard surfaces used during procedure with Trigene™ 5%, wipe dry with tissue and dispose of in incineration bin.

Switch off centrifuge, shut down Hood replacing night door.

Remove yellow bag from bin, place in second bag and close with bag tie

Replace bags in incineration bin and place sealed used bags in queue for autoclaving and then incineration; ensure timely treatment and disposal

### EXAMPLE 12

### Handling and Preparation of Urine Samples

Urine samples are preferably prepared as detailed in this example. Urine samples may then be assayed directly (liquid detection) or by converting into a solid phase sample (e.g. by blotting as in example 9).

Urology Biobank Protocol for Urine Sample Acquisition

Urine sample collection should only be undertaken in the following circumstances, (or alternative appropriate local ethical requirements):

Samples should only be obtained from the patients following detailed explanation of the purpose of the study.

The study specific consent form should be completed and signed by the patient or his/her representative.

Staff should be confident & competent on Local Guidelines for specimen handling.

The following general principles should be adhered to when handling urine samples:

All cuts & abrasions should be covered with a waterproof dressing.

Hands should be washed regularly and in between handling of different specimens.

Equipment: The urine should be voided & collected in a 150 ml Sterilin aseptic sample pot.

The sample should have Urinalysis using Bayer Multistix 10 SG, and the result documented.

Urine Specimens Required

The following urine specimens & volume will be taken. One or more may be omitted as per study protocol:

Sample must not be the first void of the morning.

Minimum volume acceptable: 50 ml.

Split this into: 20 ml Straight Urine; 30 ml+1 Protease Inhibitor tablet.

If a second void can be collected, centrifuge and split this into: Cellular Pellet+Supernatant.

Labelling of Urine Specimens

All tubes should be labelled with:

Study ID number.

The patients Date of Birth.

Initials of the patient.

The date & time the sample was taken.

Duplicate information must be written down onto a separate sheet of paper & placed with sample in individual specimen bags.

Processing Initial Void

Immediately decant off 20 mls as Straight Urine into a plastic tube.

Add Protease Inhibitor Tablet to remaining (30 ml) void, swirl to dissolve, and transfer to plastic tube.

Processing Second Void

Centrifuge immediately @ 3000 RPM for 10 minutes.

Decant off supernatant into a plastic tube & keep.

Keep Cellular Pellet.

All samples should be processed & frozen at −80° C. within 1 hour of being voided.

The health & safety guidelines pertaining to the particular workplace should be adhered to; local COSHH guidelines should be followed.

A new pipette should be used for each sample to prevent cross contamination.

The initial sample pot used for collecting the void should be discarded safely into a sharps bin.

Protein may be extracted/concentrated from the urine for analysis, for example if it is too dilute in the sample.

### EXAMPLE 13

### Stability of GINS Proteins

Samples of preferred GINS protein Sld5 and prior art marker Mcm2 were prepared.

These samples were treated with protease.

Different samples were treated with increasing concentrations of protease. One sample was untreated as a control.

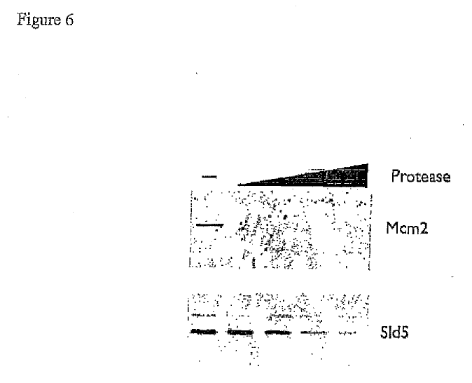

The liquid samples were then converted to solid phase samples by size-separation and Western blotting. The resulting blot was then probed with reagents for detection of Mcm2 and Sld5. The result is shown in FIG. 6. This demonstrates the enhances stability of GINS compared with MCM.

All publications mentioned in the above specification are herein incorporated by reference. Various modifications and variations of the described methods and systems of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for detecting an actively cycling ell in a sample, aid method comprising determining the state of PSF1 or SLD5 gene expression within said cell, wherein detection of PSF1 or SLD5 gene expression in said ell indicates that said cell is actively cycling.

2. A method for detecting an actively cycling cell in a subject, said method comprising assaying a sample from said subject for evidence of PSF1 or SLD5 gene expression, wherein detection of PSF 1 or SLD5 gene expression in said sample indicates that said subject comprising an actively cycling cell.

3. A method according to claim 1 wherein the gene expression is SLF5 gene expression.

4. A method according to claim 1 wherein the gene expression is PSF1 gene expression.

5. A method according to claim 1 further comprising the step of determining the state of MCM gene expression within said cell or sample, wherein detection of MCM agene expression indicates the presence of an actively cycling cell.

6. A method according to claim 1 wherein the sample is a body fluid and the method comprises detecting PSF1 or SLD5 protein within said body fluid.

7. A method according to claim 1 wherein PSF1 or SLD5 gene expression is determined by detection of PSF1 or SLD5 protein.

8. A method accrding to claim 7 wherein the PSF1 or SLD5 protein is detected by immunochemistry.

9. A method according to claim 1 wherein the detection is performed on a liquid sample.

10. A method according to claim 1 wherein the PSF1 or the SLD5 is extracellular.

11. A method of identifying proliferating or non-proliferating cells in a sample said method comprising determining the state of PSF1 or SLD5 expression within said ells, wherein detection of PSF1 or SLD5 expression in a cell indicates that said cell proliferating, and absence of PSF1 or SLD5 expression in a cell indicate that said ell is non-proliferating.

12. A method according to claim 11 wherein both proliferating and non-proliferating cells are detected in a single sample.

13. A method of determining the phase of the cell which a cell is in, comprising determining the level of PSF1 or SLD5 protein in a said cell wherein an enhanced level of PSF1 or SLD5 protein in said cell, wherein an enhanced level of PSF1 or SLD5 protein indicates that said cell is in S-phase.
